(19) United States
(12) Patent Application Publication
(10) Pub. No.: US 2005/0180995 A1
(43) Pub. Date: Aug. 18, 2005

(54) Method to Improve Grafted Hair Growth And Retain Grafted Hair

(76) Inventors: Brian Freund, Pickering (CA); Marvin Schwartz, Pickering (CA)

Correspondence Address: BRIAN FREUND, 944 MERRITTON RD., PICKERING, ON, L1V1B1, CA

(21) Appl. No.: 10/906,321

(22) Filed: Feb. 14, 2005

Related U.S. Application Data
(60) Provisional application No. 60/521,081, filed on Feb. 18, 2004.

(51) Int. Cl. A61K 39/08

(52) U.S. Cl. 424/239.1

(57) ABSTRACT

This invention is a method for improving the growth and retention of any type of hair graft or hair transplant on the scalp of a human patient. The method comprises the step of administering a presynaptic neurotoxin into the scalp of the patient in a quantity and concentration to provide a therapeutically-effective flaccid paralysis of the muscle tissue outside of the skull of the patient. The result is a reduction in tissue tension in the desired area of hair-growth without affecting any tissue not outside the skull of the patient.

### BACKGROUND OF INVENTION

The present invention relates to a method of treating the scalp to improve the growth of grafted hair and retention of grafted hair on the scalp.

The loss of scalp hair is a problem that plagues men and some women. The effects of this condition can have significant physical, economic and psychological impact. Great effort and expense has been spent trying to prevent and reverse it.

It is now generally appreciated that there are several discernible categories, of hair loss and, logically flowing from the distinctions of the different categories, that each category in and to itself need be separately considered if hair loss reduction or re-growth is desired by any particular person suffering from hair loss.

The most common type of hair loss is androgenetic alopecia. In men this is referred to as male pattern baldness. Usually with alopecia, hair loss happens gradually over many years. It starts out being most obvious on the crown of the head and in the frontal region in men. For women afflicted with alopecia, the hair loss, which provides a thinning effect, is more spread out and is common after menopause. Despite this type of hair loss being common, it is one of the categories that has met with only very limited success on the part of scalp and hair specialists in being able to reverse its effect.

The cause of the hair loss is thought to be due to a combination of one or more mechanisms. Identified mechanisms include poor nutrition to the hair follicles, reduced oxygen supply to the follicles and an increase in the levels of androgenic or male hormones in the regions which are loosing hair. These mechanisms are directly influenced by blood flow to the scalp via peripheral blood vessels.

As a result, in the vast majority of cases, effective treatment may be limited to one or another form of hair graft or hair transplantation surgery.

Hair transplantation has been used for many years to improve thinning or balding areas of the scalp. There are many different surgical techniques which in general involve the movement of hair bearing tissue from one area of the scalp to another. Most commonly, hair is harvested from the back of the scalp in strips and cut into small sections or micrografts containing several hairs and their follicals. These grafts are then inserted into donor sites in the balding areas of scalp. Although the grafts have with them their own blood vessels which feed the hair follicals, these small vesels must attach to the existing network of vessels which may be inadequate to properly sustain the grafted hair.

As a result there is an immediate loss of some of grafted hair during the healing phase as well as the eventual generalized loss of grafts due to the underlying inadequate blood flow to the scalp.

It is an object of the present invention to provide a method of enhancing the quality, quantity and longevity of transplanted hair in both men and women suffering from alopecia that does not require external application, that does not require extensive effort of the user and which is economical to use.

### SUMMARY OF THE INVENTION

According to an aspect of this invention there is provided a method for improving hair graft growth and retention on the scalp of a human patient comprising the step of administering a presynaptic neurotoxin into the scalp of the patient in a quantity and concentration to provide a therapeutically-effective flaccid paralysis of the muscle tissue outside of the skull of the patient to reduce tissue tension in the desired area of graft growth without affecting any tissue not outside the skull of the patient.

The invention will be more readily understood after reading the following detailed description of the preferred embodiment of the invention in which:

There is the administration of a presynaptic neurotoxin by a qualified health care worker, using a hypodermic needle to the scalp of the patient, in a quantity and concentration, as will be more fully described below, to provide a therapeutically-effective flaccid paralysis of the muscle tissue outside of the skull of the patient to reduce tissue tension in the desired area of graft-growth without affecting any tissue not outside the skull.

Preferably, the presynaptic neurotoxin used is a botulinum toxin, namely botulinim toxin A. Preferably, it is injected, after being reconstituted with normal saline or other suitable and pharmaceutically acceptable carrier or diluent to achieve the desired concentration and volume of injectant, into the frontal muscles, into the temporal muscles, and into the occipital muscles in a circumferential manner around the periphery of the scalp. It will be appreciated by those skilled in the art that the peri-auricular group includes the superior auricular muscle as well as the posterior auricular muscle and the anterior portions of the occipital muscle.

The injections are placed to the areas of the scalp as required for treating the graft donor site. The anatomy of the scalp can be conceptualized tension-wise as trampoline-like. The layers beneath the skin are an unyielding fibrous layer called an aponeurosis (galea aponeurotica). The muscles surrounding the scalp are analogous to the springs which would tension and hold taut the tarp of the trampoline. Blood vessels carrying oxygen and nutrients must traverse this area to provide a suitable environment for the grafted hair follicles to flourish. When the muscles surrounding the scalp tighten, the areas of scalp mechanically under the highest tension are the vertex and frontal areas. Thus, the most common areas of hair loss correspond to the areas under highest tension. The inventors have found that by reducing the tension of the musculature surrounding the scalp, tension in the tissue of the frontal and vertex areas is also effectively reduced. This reduction of tension in the frontal and vertex areas permits an increase of blood flow into that area of the scalp. So it is that the ring of musculature surrounding the scalp controls the tension in the target areas. When it is relaxed, the tissue of the whole scalp relaxes.

So it is in the present invention that a complete circumferential distribution of the toxin around the periphery of the scalp of the person suffering from hair loss is preferable so that a complete circumferential relaxation the muscle areas responsible for tensioning the underlying tissue of the grafted areas is achieved.

The object of the injections is to reduce scalp tension. The relative contribution of these muscles to scalp tension will vary in different individuals and, accordingly, it would not be necessary in some individuals to target all these muscle groups to reduce scalp tension. The relative contribution and therefore importance can be readily determined through clinical assessment. Specific factors to be considered would be apparent to the competent health care worker and depend upon the specific patient, such factors would include scalp muscle bulk and the muscle's ability to move the scalp when contracted voluntarily or by muscle point stimulation.

Diluted botulinum toxin A, the preferred presynaptic neurotoxin, can be deposited in an extra muscular site close to the target muscle but preferably is deposited within the target muscles bodies. It is injected preferably with a fine gauge hypodermic needle or EMG needle but may also be delivered by other modes, acceptable in the art, such as passive absorption, iontophoresis or pneumatic techniques. The muscles to be affected by administration of presynaptic neurotoxin can be determined or confirmed through digital palpation or through electromyographic guidance.

Subject to the specific situation of the patient and the botulinum toxin used, generally each patient can receive injections of approximately 0.1 ml of botulinum toxin-A diluted to 10 units per 0.1 ml in a pattern whereby a radius of 1.5 cm drawn from the center of the injection site overlaps a similar radius of the adjacent injection site. Reasonably, a larger volume of or higher concentration of botulinum toxin-A will allow for more space between the injection sites. The adequacy of the injection technique can be monitored through an objective increase in scalp mobility through digital pressure or through an increase in transcutaneous partial oxygen pressure measurements. A reading taken in the graft recipient site which equals or approaches that of a standardized hair bearing area such as the temporal scalp can be taken as indicative of an adequate response to the technique and therefore adequate performance of the technique.

The administration of the presynaptic neurotoxin produces a reversible, flaccid paralysis of muscle tissue very local to the area of injection which should remain for a period of three to nine months, if properly administered. The procedure need only be repeated periodically as necessary to maintain laxity of the scalp muscles necessary to permit graft hair growth. The process therefore requires relatively little effort of the patient.

The method described is well suited to any type of hair grafting procedure including follicular cloning techniques and the placement of pre-follicular tissue grafts.

It will be appreciated by those skilled in the art that chemicals used as toxins in humans for health purposes must be empirically tested before they can be readily used by the public or by health care workers. The properties of any given chemical in a particular situation cannot be predicted with absolute certainty without proper testing. Although the qualities of the toxin required to achieve the results of the present invention are known and described herein, there is only at this time one toxin known to work and that has been tested. As described in the preferred embodiment, that toxin is botulinim toxin-A manufactured by Allergan Inc. of Irvine Calif. However, other toxins which would imitate botulinim toxin A's qualities in paralyzing muscle tissue safely could also be used. It is the qualities of botulinim toxin-A that are desired for this invention and if a substitute toxin with the necessary qualities, as claimed in the broadest claim herein, was to become available, it would be apparent to a person skilled in the art as falling within the scope of this invention.

The health the grafted hair follicles are chiefly governed by factors such as local oxygen supply, nutrients and biochemical mediators such as hormones. The relative concentration of these factors as well as the biochemical reactions they are involved in is quite dependent on the blood supply to the tissues. By using the arterial vessels as described above, blood supply is provided to the central areas of the scalp from the periphery. The inventors have found that injection of a presynaptic neurotoxin as described reduces scalp tension and, by doing so, improves bloodflow through the arteries. In turn, oxygen and nutrient levels improve while the level of the male hormone dihydrotestosterone, which inhibits follicular development, is reduced. Ultimately hair graft growth and retention is improved.

For illustrative purposes in using the invention, the following is an actual case study in the use of the invention: A healthy 44 year old non-smoker with Hamilton class V male pattern baldness underwnent a micro-graft hair replacement surgey to the frontal area. After 6 months it was noted that approximately 40% of the grafted hair failed to grow and subsequently was lost. Prior to a second grafting proceedure the subject was injected intramuscularly with 100 units botulinum toxin-A (Botox^{R}, Allergan Inc. Irvine, Calif.) diluted to 10 units per 0.1 ml in normal saline. The frontal, temporal, peri-auricular and occipital muscles were injected with a fine gauge hypodermic needle in a circumferential manner around the periphery of the scalp as described above. Based on serial photography, the second grafts placed after treatment of the scalp showed significanly greater growth and retention in a six month time frame. The above example is given to show actual results achieved using this invention and is given for illustrative purposes of its effect.

It will be apparent to those skilled in the art reading this specification as a whole that there are alternatives to following the exact procedure as outlined in the method described as the preferred embodiment herein without deviating from the scope of the invention. It is not intended that the present specification be read in a limiting manner but that the scope of the invention be appreciated as set out in the appended claims.

1. A method for improving the growth and retention of any type of hair graft or hair transplant on the scalp of a human patient comprising the step of administering a presynaptic neurotoxin into the scalp of the patient in the area of the muscular band extending around the scalp defined by the frontal muscles, temporal muscles, peri-auricular muscles and occipital muscles in a quantity and concentration to provide a therapeutically effective flaccid paralysis of the muscle tissue outside of the skull of the patient to reduce tissue tension inside the area of the muscular band to thereby reduce tissue tension in the grafted area on the scalp the perimeter of which is defined by the muscular band without affecting any tissue not outside the skull of the patient.

2. A method for improving the growth and retention of any type of hair graft or hair transplant on the scalp of a human patient as claimed in claim 1 wherein the presynaptic neurotoxin is a botulinum toxin.

3. A method for improving the growth and retention of any type of hair graft or hair transplant on the scalp of a human patient as claimed in claim 1 wherein the presynaptic neurotoxin is botulinum toxin A.

4. A method for improving the growth and retention of any type of hair graft or hair transplant on the scalp of a human patient as claimed in claim 1 wherein the presynaptic neurotoxin is administered by injection with a hypodermic needle.

5. A method for improving the growth and retention of any type of hair graft or hair transplant on the scalp of a human patient as claimed in claim 2 wherein the presynaptic neurotoxin is administered by injection with a hypodermic needle.

6. A method for improving the growth and retention of any type of hair graft or hair transplant on the scalp of a human patient as claimed in claim 3 wherein the presynaptic neurotoxin is administered by injection with a hypodermic needle.

7. A method for improving the growth and retention of any type of hair graft or hair transplant on the scalp of a human patient as claimed in claim 1 wherein the presynaptic neurotoxin is administered by injection with a hypodermic needle in a circumferential manner around the periphery of the scalp.

8. A method for improving the growth and retention of any type of hair graft or hair transplant on the scalp of a human patient as claimed in claim 2 wherein the presynaptic neurotoxin is administered by injection with a hypodermic needle in a circumferential manner around the periphery of the scalp.

9. A method for improving the growth and retention of any type of hair graft or hair transplant on the scalp of a human patient as claimed in claim 3 wherein the presynaptic neurotoxin is administered by injection with a hypodermic needle in a circumferential manner around the periphery of the scalp.

10. A method for improving the growth and retention of any type of hair graft or hair transplant on the scalp of a human patient as claimed in claim 1 wherein the presynaptic neurotoxin is administered to the frontal, temporal per-auricular and occipital muscles of the patient.

11. A method for improving the growth and retention of any type of hair graft or hair transplant on the scalp of a human patient as claimed in claim 2 wherein the presynaptic neurotoxin is administered to the frontal, temporal peri-auricular and occipital muscles of the patient.

12. A method for improving the growth and retention of any type of hair graft or hair transplant on the scalp of a human patient as claimed in claim 3 wherein the presynaptic neurotoxin is administered to the frontal, temporal peri-auricular and occipital muscles of the patient.
